(19) United States
(12) Patent Application Publication
(10) Pub. No.: US 2026/0224451 A1
(43) Pub. Date: Aug. 6, 2026

(54) MAGNESIUM BASED PERSONAL CARE PRODUCTS SUBSTANTIALLY DEVOID OF ENDOCRINE DISRUPTING CHEMICALS

(71) Applicant: Go BRIXY, Inc., La Jolla, CA (US)

(72) Inventor: Kevin Brodwick, Park City, UT (US)

(21) Appl. No.: 19/298,781

(22) Filed: Aug. 13, 2025

Related U.S. Application Data
(60) Provisional application No. 63/753,003, filed on Feb. 3, 2025.

(51) Int. Cl. A61K 8/19; A61Q 15/00

(52) U.S. Cl. CPC A61K 8/19; A61Q 15/00; A61K 2800/30

(57) ABSTRACT

Provided herein are methods and compositions for personal care products including magnesium hydroxide and that are substantially free of endocrine disrupting chemicals.

### CROSS-REFERENCE TO RELATED APPLICATIONS

The present application claims priority to U.S. provisional application Ser. No. 63/753,003, filed Feb. 3, 2025, herein incorporated by reference in its entirety.

### BACKGROUND

The invention generally relates to personal care products, specifically personal care products that are substantially free of endocrine disrupting chemicals.

Current products on the market consist predominantly of aluminum and sodium bicarbonate. There are health concerns associated with both. Primarily the potential for increased risk of breast cancer and endocrine disruption with aluminum and some portion of the population has sensitivity to sodium bicarbonate.

Endocrine disruptors may act as antagonists or antagonists to estrogens or androgens and may interfere in multiple ways with the action of thyroid hormones. For example, endocrine disruptors can mimic or block chemicals naturally found in the body, thereby altering the body's ability to produce hormones, interfering with the ways hormones travel through the body, and altering the concentration of hormones reaching hormone receptors. Although relatively few chemicals have been examined for their endocrine disruptive effect, those chemicals that have shown such an effect are increasingly found in common food, plastic, and personal care products.

Endocrine disruptors include chemicals such as, for example, polychlorinated biphenyls (PCBs), dioxins and furans. These chemicals are found environmentally in insecticides, herbicides, fumigants, and fungicides that are used in agriculture as well as in the home. Other endocrine disruptors are found in common chemicals such as detergents, dental amalgams and resins that coat the inside of tin cans. In addition, research has found that, because the polymerization process used to make plastics is not complete, unpolymerized monomers with estrogenic activity can migrate out of plastics resulting in deleterious estrogenic, carcinogenic or toxic effects in humans and other species.

Di(2-ethylhexyl) phthalate (DEHP) is a plasticizer that is a colorless liquid with almost no odor. DEHP is commonly used in producing polyvinyl chloride (PVC) plastic products like toys, vinyl upholstery, shower curtains, adhesives, and coatings. PVC plastic may contain up to 40% DEHP. DEHP, an estrogen-mimicking chemical, has been found to leach from these plastics, thereby creating problems with the materials in contact with the plastic. For example, DEHP has been found to leach from intravenous bags and tubing into the blood and medications being administered to patients. Exposure to DEHP through intravenous bags and tubing presents a health risk that could be avoided through the use of a plastic material that does not contain endocrine disruptive chemicals.

Similarly, bisphenol A is a monomer used to synthesize a number of plastics, such as polycarbonates, epoxy, phenoxy and polysulfone polymers, and is released in significant amounts when these plastics are exposed to water, particularly when heated. Bisphenol A has been shown to possess deleterious estrogenic activity. Nevertheless, plastics manufactured with bisphenol A, such as polycarbonate, are commonly used for food and beverage containers, baby bottles, baby toys, microwaveable containers and medical items.

The association between the exposure and bioaccumulation of endocrine disruptor chemicals (EDCs) and their adverse effects on human and wild life populations has raised concern worldwide. Some examples of the effects of EDCs are: decreased reproductive success and feminization of males in several wildlife species; increased hypospadias along with reductions in sperm counts in men; increase in the incidence of human breast and prostate cancers; and endometriosis. Because these chemicals are ubiquitous, highly lipophilic, and often chlorinated, this ensures their persistent presence in the environment resulting in their bioaccumulation in the food chain.

Some compounds used in personal care products, such as deodorant, are absorbed and stored in fat cells, which are prevalent in the underarm area. Underarm tissue also contains hormone receptors, which could react to some of those same deodorant ingredients. Chemicals placed on the skin can enter the bloodstream without being metabolized. Blood tests in recent research show that many of the substances commonly included in deodorant products can get past the epidermis and into the body. Certain compounds in antiperspirant and deodorant could cause or contribute to developmental or reproductive issues, as well as cancer. There are at least five deodorant ingredients of concern, in particular: parabens, aluminum, triclosan, phthalates, and fragrances.

Parabens are used as preservatives in deodorant and other personal care products. Research suggests some parabens may interfere with the way your body produces and regulates estrogen and other hormones. For example, the breast includes estrogen-sensitive tissue. A concern of placing parabens close to this tissue every day is that the parabens may promote the growth of cancer cells, and in both men and women. Research has shown that mixing different parabens can strengthen their estrogenic effects. Existing evidence suggests that long-term, low-dose mixtures of environmental chemicals—including parabens—could cause cancer.

Typically found only in antiperspirants, aluminum can cause gene instability in breast tissue, research shows. This instability can cause changes than may promote the growth of tumors or cancer cells. Over 50% of breast cancers start in the upper outer quadrant of the breast local to the underarm region. Breast cancer incidence tends to align with use of products that contain aluminum. The situation is made worse by applying a product containing aluminum to an area that is frequently shaved had may have broken skin.

Cosmetic manufacturers add triclosan to many products in order to prevent bacterial contamination, and to kill bacteria on the surface of the skin, as in anti-acne products, some deodorants and antiperspirants, and in sanitizing hand soaps. Triclosan is so common that 75% of Americans have detectable levels of the stuff in their urine.

Some animal studies have linked triclosan to unusual hormone activity. More research suggests triclosan could mess with your microbiome or the day-to-day operations of your genes. There is evidence from amphibians and fish is that triclosan impairs thyroid function, which is crucial for brain development. Blood tests show triclosan is at the high end when it comes to chemicals found in people's bodies.

Phthalates help deodorant and other cosmetics—such as fragrance—stick to your skin. They also appear to disrupt “androgen function,” or the way your body produces and uses the hormone testosterone. Testosterone, found in both men and women, plays a role in energy and muscle maintenance. Phthalates could impair reproductive ability in men. Phthalates could impact fetal development in pregnant women. Research has also linked phthalates to lower IQs and higher rates of asthma. Phthalates are typically present in any product with a fragrance that lingers after it's been used or applied; phthalates are partly what makes smells stick. That means that everything from body wash to shampoo to lotion to hairspray to soap.

Almost every scented product has fragrance or perfume listed among its ingredients. It is difficult to know what specific chemicals are included in the terms “fragrance” or “perfume” because scents are protected under trade law. The terms could refer to phthalates, or they could be substances that cause allergies or skin irritation. Even smelling scented products on other people can cause an allergic reaction.

Present invention attempts to solve these problems as well as others.

### SUMMARY OF THE INVENTION

Provided herein are methods and compositions for personal care products including magnesium hydroxide and that are substantially free of endocrine disrupting chemicals.

The composition and methods are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Description of the Embodiments

Embodiments of the present invention are directed to personal care deodorant and antiperspirant products that are void of and do not contain any endocrine disrupting chemicals, and a method of making the same. An embodiment of the personal care deodorant product can be used as an underarm deodorant to reduce/prevent body odor. An embodiment of the personal care antiperspirant product can used to prevent perspiration, typically under the arm, thereby reducing/preventing the body odor resulting from underarm perspiration. Embodiments of the personal care deodorant/anti-perspirant product can be used as a substitute for traditional underarm deodorants that include chemicals that are known to have endocrine disrupting effects or are suspected of having endocrine disruptive effects.

The term “deodorant” will be used herein to refer collectively to personal care deodorant and antiperspirant products in accordance with embodiments of the present invention. The deodorant composition may further comprise other optional ingredient(s). The compositions can be in the form of a solid stick. The compositions may be substantially free of or free of a substituted or unsubstituted 2-pyridinol-N-oxide material (piroctone olamine). The deodorant composition may be anhydrous. The deodorant composition may be free of added water.

“Substantially free of” refers to about 0.2% or less, about 0.1% or less, or about 0.01% or less of a stated ingredient. “Free of” or “substantially void” refers to no detectable amount of the stated ingredient or thing.

Embodiments of the deodorant include one or more of the following: a main base ingredient substantially void of endocrine disrupters, a skin conditioning agent substantially void of endocrine disrupters, an emollient substantially void of endocrine disrupters, a nonaqueous viscosity increasing agent substantially void of endocrine disrupters, a surfactant/emulsifying agent substantially void of endocrine disrupters, an absorbent substantially void of endocrine disrupters, a soothing agent substantially void of endocrine disrupters, a deodorant agent substantially void of endocrine disrupters, a viscosity-controlling agent substantially void of endocrine disrupters, and a scent substantially void of endocrine disrupters. Where the main base ingredient is magnesium hydroxide.

Embodiments of the present invention do not include and are substantially void of parabens, aluminum, triclosan, phthalates, di(2-ethylhexyl) phthalate (DEHP), bisphenol A (BPA). In other embodiment, the present invention does not include aluminum-based formulas, zinc oxide or sodium bicarbonate. Endocrine disrupting chemicals may include chemical structures that cause estrogenic activity. Sodium bicarbonate has shown an increased risk of breast cancer and endocrine disruption with aluminum and some portion of the population has sensitivity to sodium bicarbonate. In addition, sodium bicarbonate-based solution causes the staining of clothing.

Magnesium may be in the form of magnesium hydroxide, magnesium carbonate, magnesium hydroxide & magnesium carbonate hydroxide, magnesium carbonate hydroxide, partially carbonated magnesium hydroxide, derivatives thereof, and the like.

The skin conditioning agent is used to increase water content on surface of the skin. The skin conditioning agent can include caprylic/capric triglyceride, aloe vera, allantonin, rose hip oil, beta glucan, bamboo extract, carrot cells, glycoproteins, cucumber fruit extract, grape seed extract, hyaluronic acid, mulberry root, rosemary, sage, vitamin A, and White tea extract. Caprylic/capric triglyceride, aloe vera, allantonin, rose hip oil, beta glucan, bamboo extract, carrot cells, glycoproteins, cucumber fruit extract, grape seed extract, hyaluronic acid, mulberry root, rosemary, sage, vitamin A, and White tea extract are not known to have any endocrine disrupting effects.

The emollient is used to soften the skin and increase the moisture on the skin by reducing evaporation. The emollient can include oil from Cocos nucifera (coconut oil), hemp seed oil, jojoba oil, green tea, keratin protein, oat amino acids, and sunflower oil. Cocos nucifera, hemp seed oil, jojoba oil, green tea, keratin protein, oat amino acids, and sunflower oil are not known to have any endocrine disrupting effects.

The nonaqueous viscosity-increasing agent is used to thicken of the liquid (oil) portion of cosmetic formulations. The nonaqueous viscosity-increasing agent can include wax from Euphorbia cerifera (Candelilla), polyethylenes, trihydroxystearin, organoclays, fumed silica. Euphorbia cerifera (Candelilla) wax, polyethylenes, trihydroxystearin, organoclays, fumed silica are not known to have any endocrine disrupting effects.

The aqueous viscosity-increasing agent is used to thicken of the liquid (water) portion of cosmetic formulations. The aqueous viscosity-increasing agent can include cellulose gum, guar gum, xanthan gum, hydroxyethylcellulose, methylcellulose, polyethylene glycols, and clays. Cellulose gum, guar gum, xanthan gum, hydroxyethylcellulose, methylcellulose, polyethylene glycols, clays are not known to have any endocrine disrupting effects.

The surfactant/emulsifying agent is used to lower the surface tension between two liquids or a liquid and a solid. The surfactant/emulsifying agent can include wax from Cera alba (beeswax), sulfate, sulfonate, phosphate, and carboxylates, ammonium lauryl sulfate, sodium lauryl sulfate (SLS), sodium dodecyl sulfate (SDS), alkyl-ether sulfates, sodium laureth sulfate, sodium lauryl ether sulfate (SLES), sodium myreth sulfate, sodium stearate, sodium lauroyl sarcosinate, cocamidopropyl betaine, Lauryl glucoside, Glyceryl laurate, Cocamide MEA, and cocamide DEA. Cera alba wax (beeswax), sulfate, sulfonate, phosphate, and carboxylates, ammonium lauryl sulfate, sodium lauryl sulfate (SLS), sodium dodecyl sulfate (SDS), alkyl-ether sulfates, sodium laureth sulfate, sodium lauryl ether sulfate (SLES), sodium myreth sulfate, sodium stearate, sodium lauroyl sarcosinate, cocamidopropyl betaine, Lauryl glucoside, Glyceryl laurate, Cocamide MEA, and cocamide DEA are not known to have any endocrine disrupting effects.

The absorbent is used to absorb perspiration that can cause body odor, particularly in the underarm area. The absorbent can include starch from Zea mays (corn starch), arrowroot powder, sodium stearate, sodium bicarbonate, charcoal, and dextrin. Zea mays (corn starch), arrowroot powder, sodium stearate, sodium bicarbonate, charcoal, and dextrin are not known to have any endocrine disrupting effects.

The soothing agent is used to provide care to irritated or stressed skin or to reduce the potential for irritated or stressed skin. The soothing agent can include Maranta arundinacea (arrowroot) powder, caprylic/capric triglyceride, aloe vera, allantonin, rose hip oil, beta glucan, bamboo extract, carrot cells, glycoproteins, cucumber fruit extract, grape seed extract, hyaluronic acid, mulberry root, rosemary, sage, vitamin A, and White tea extract. Maranta arundinacea (arrowroot) powder, caprylic/capric triglyceride, aloe vera, allantonin, rose hip oil, beta glucan, bamboo extract, carrot cells, glycoproteins, cucumber fruit extract, grape seed extract, hyaluronic acid, mulberry root, rosemary, sage, vitamin A, and White tea extract are not known to have any endocrine disrupting effects.

The deodorant agent is used to reduce or eliminate body odor in and around the area where the deodorant is applied. The deodorant agent can include magnesium hydroxide.

The viscosity-controlling agent is used to control the thickness of aqueous and nonaqueous cosmetic formulation. The viscosity-controlling agent can include Butyrospermum parkii (Shea) butter, cera alba wax (beeswax), sulfate, sulfonate, phosphate, and carbo, xylates, ammonium lauryl sulfate, sodium lauryl sulfate (sodium dodecyl sulfate, SLS, or SDS, alkyl-ether sulfates sodium laureth sulfate (sodium lauryl ether sulfate or SLES), and sodium myreth sulfate, sodium stearate, sodium lauroyl sarcosinate, cocamidopropyl betaine, Lauryl glucoside, Glyceryl laurate, Cocamide MEA, and cocamide DEA. Butyrospermum parkii (Shea) butter, cera alba wax (beeswax), sulfate, sulfonate, phosphate, and carbo, xylates, ammonium lauryl sulfate, sodium lauryl sulfate (sodium dodecyl sulfate, SLS, or SDS, alkyl-ether sulfates sodium laureth sulfate (sodium lauryl ether sulfate or SLES), and sodium myreth sulfate, sodium stearate, sodium lauroyl sarcosinate, cocamidopropyl betaine, Lauryl glucoside, Glyceryl laurate, Cocamide MEA, and cocamide DEA are not known to have any endocrine disrupting effects.

Scent is used to give the product a pleasing smell and to mask any residual body odor that is not entirely eliminated by the deodorant agent. Scent can include currant, Citrus paradisi (grapefruit), Essential oils, fruit extract, fruit oil, flower oil, flower powder, flower extract. Currant, Citrus paradisi, Essential oils, fruit extract, fruit oil, flower oil, flower powder, and flower extract are not known to have any endocrine disrupting effects.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Juniper Journey deodorant

Zea Mays (Corn) Starch, Caprylic/Capric Triglyceride, Magnesium Hydroxide, Euphorbia Cerifera (Candelilla) Wax, Diatomaceous Earth, Cocos Nucifera (Coconut) Oil, Lauryl Laurate, Polyhydroxystearic Acid, Butyrospermum Parkii (Shea) Butter, Stearyl Stearate, Triethyl Citrate, Jojoba Esters, Dipteryx Odorata (Tonka Bean) Seed Extract, Dimethyl Heptenal, Juniperus Communis (Common Juniper) Fruit Oil, Juniperus Mexicana Oil, Linalyl Acetate, Rosmarinus Officinalis (Rosemary) Leaf Oil, Vetiveria Zizanoides (Vetiver) Root Oil, Niacinamide, Cetearyl Alcohol, Ceramide NP, Limonene, Coumarin, Linalool.

### Vanilla Vacay Deodorant

Zea Mays (Corn) Starch, Caprylic/Capric Triglyceride, Magnesium Hydroxide, Euphorbia Cerifera (Candelilla) Wax, Diatomaceous Earth, Cocos Nucifera (Coconut) Oil, Lauryl Laurate, Triethyl Citrate, Polyhydroxystearic Acid, Butyrospermum Parkii (Shea) Butter, Stearyl Stearate, Jojoba Esters, Gamma-Nonalactone, Niacinamide, Cetearyl Alcohol, Vanillin, Pogostemon Cablin (Patchouli) Oil, Ceramide NP

### Plumeria Crush Deodorant

Zea Mays (Corn) Starch, Caprylic/Capric Triglyceride, Magnesium Hydroxide, Euphorbia Cerifera (Candelilla) Wax, Diatomaceous Earth, Cocos Nucifera (Coconut) Oil, Lauryl Laurate, Triethyl Citrate, Polyhydroxystearic Acid, Butyrospermum Parkii (Shea) Butter, Stearyl Stearate, Jojoba Esters, Gamma Octalactone, Niacinamide, Cetearyl Alcohol, 2-Phenylethanol, Amyris Balsamifera (Amyris) Bark Oil, Gamma-Undecalactone, Linalyl Acetate, Litsea Cubeba Fruit Oil, Vanillin, 3-Hexenol, Benzyl Acetate, Ceramide NP, Decanal, Juniperus Mexicana Oil, Raspberry Ketone, Citral, Hydroxycitronellal, Limonene

### Almond Blossom Deodorant

Zea Mays (Corn) Starch, Caprylic/Capric Triglyceride, Magnesium Hydroxide, Euphorbia Cerifera (Candelilla) Wax, Diatomaceous Earth, Cocos Nucifera (Coconut) Oil, Lauryl Laurate, Triethyl Citrate, Polyhydroxystearic Acid, Butyrospermum Parkii (Shea) Butter, Stearyl Stearate, Jojoba Esters, Niacinamide, Cetearyl Alcohol, Geranyl Acetate, Heliotropine, Phenethyl Alcohol, Pogostemon Cablin (Patchouli) Oil, Prunus Amygdalus Amara (Bitter Almond) Kernel Oil, Ceramide NP, Lavandula Angustifolia (Lavender) Oil, Linalool.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A personal care deodorant product substantially void of any endocrine disrupting chemicals, the personal care deodorant product comprising:
a. a skin conditioning agent substantially void of any endocrine disrupting chemicals;
b. an emollient substantially void of any endocrine disrupting chemicals;
c. a nonaqueous viscosity increasing agent substantially void of any endocrine disrupting chemicals;
d. an aqueous viscosity increasing agent substantially void of any endocrine disrupting chemicals;
e. a surfactant/emulsifying agent substantially void of any endocrine disrupting chemicals;
f. an absorbent substantially void of any endocrine disrupting chemicals;
g. a soothing agent substantially void of any endocrine disrupting chemicals;
h. a deodorant agent based on magnesium substantially void of any endocrine disrupting chemicals;
i. a viscosity-controlling agent substantially void of any endocrine disrupting chemicals; and
j. a scent substantially void of any endocrine disrupting chemicals.

2. The personal care deodorant product of claim 1, comprising: Zea Mays (Corn) Starch, Caprylic/Capric Triglyceride, Magnesium Hydroxide, Euphorbia Cerifera (Candelilla) Wax, Diatomaceous Earth, Cocos Nucifera (Coconut) Oil, Lauryl Laurate, Triethyl Citrate, Polyhydroxystearic Acid, Butyrospermum Parkii (Shea) Butter, Stearyl Stearate, Jojoba Esters, Gamma-Nonalactone, Niacinamide, Cetearyl Alcohol, Vanillin, Pogostemon Cablin (Patchouli) Oil, and Ceramide NP.

3. The personal care deodorant product of claim 1, comprising: Zea Mays (Corn) Starch, Caprylic/Capric Triglyceride, Magnesium Hydroxide, Euphorbia Cerifera (Candelilla) Wax, Diatomaceous Earth, Cocos Nucifera (Coconut) Oil, Lauryl Laurate, Triethyl Citrate, Polyhydroxystearic Acid, Butyrospermum Parkii (Shea) Butter, Stearyl Stearate, Jojoba Esters, Gamma Octalactone, Niacinamide, Cetearyl Alcohol, 2-Phenylethanol, Amyris Balsamifera (Amyris) Bark Oil, Gamma-Undecalactone, Linalyl Acetate, Litsea Cubeba Fruit Oil, Vanillin, 3-Hexenol, Benzyl Acetate, Ceramide NP, Decanal, Juniperus Mexicana Oil, Raspberry Ketone, Citral, Hydroxycitronellal, and Limonene.

4. The personal care deodorant product of claim 1, comprising: Zea Mays (Corn) Starch, Caprylic/Capric Triglyceride, Magnesium Hydroxide, Euphorbia Cerifera (Candelilla) Wax, Diatomaceous Earth, Cocos Nucifera (Coconut) Oil, Lauryl Laurate, Triethyl Citrate, Polyhydroxystearic Acid, Butyrospermum Parkii (Shea) Butter, Stearyl Stearate, Jojoba Esters, Niacinamide, Cetearyl Alcohol, Geranyl Acetate, Heliotropine, Phenethyl Alcohol, Pogostemon Cablin (Patchouli) Oil, Prunus Amygdalus Amara (Bitter Almond) Kernel Oil, Ceramide NP, Lavandula Angustifolia (Lavender) Oil, and Linalool.
